(19) United States
(12) Patent Application Publication
(10) Pub. No.: US 2018/0002820 A1
(43) Pub. Date: Jan. 4, 2018

(54) COMPOSITION AND METHOD FOR INHIBITING CORROSION OF METALS

(71) Applicant: BAKER HUGHES INCORPORATED, HOUSTON, TX (US)

(72) Inventors: ZHENGWEI LIU, HOUSTON, TX (US); TRACEY S. JACKSON, SUGAR LAND, TX (US); JACOB GIBSON, CALGARY (CA)

(73) Assignee: BAKER HUGHES INCORPORATED, HOUSTON, TX (US)

(21) Appl. No.: 15/198,347

(22) Filed: Jun. 30, 2016

(51) Int. Cl. C23F 11/04; C07D 235/28

(52) U.S. Cl. CPC C23F 11/04; C07D 235/28

(57) ABSTRACT

A composition for inhibiting corrosion of a metal is provided. The corrosion inhibitor composition comprises 5-amino-2-mercaptobenzimidazole. The corrosion inhibitor composition outperforms many other sulfur-containing corrosion inhibitors in kettle testing.

### BACKGROUND

Metals and metal alloys are subject to corrosion in certain oil and gas production environments. These metals and metal alloys may be exposed to a variety of acids, bases, acid gases such as CO₂ and H₂S, brines of various salinities, and/or corrosive chemicals. The result of these interactions is usually formation of an oxide and/or a salt of the original metal. In most cases, corrosion causes the dissolution of the material.

It is known to treat these metals and metal alloys with corrosion inhibitors to combat corrosion. For example, 2-mercaptobenzimidazole (2-MEBI) is known to show good corrosion inhibition capability on mild steels in both strong acid and weak CO₂ saturated solutions. However, the inhibition efficiency of 2-MEBI is unable to achieve superior protection (where % protection is defined as the blank corrosion rate minus the inhibited rate and the result is divided by the blank inhibited rate and then multiplied by 100%) at reasonable dosages (less than 3000 ppm). In addition when this intermediate is used in product formulations, the end product also requires high dosages to achieve >95% protection.

Improvements in this field of technology are therefore desired.

### SUMMARY

Various illustrative embodiments of a method of inhibiting corrosion of a metal are disclosed herein. In certain aspects, the metal can be contacted with a corrosion inhibitor composition comprising an effective amount of 5-amino-2-mercaptobenzimidazole. In certain aspects, the metal can be contacted with an effective amount of a corrosion inhibitor composition comprising 5-amino-2-mercaptobenzimidazole. The metal can be carbon steel. The corrosion inhibitor composition can be contacted with the metal in an environment which would otherwise be corrosive to the metal. The environment can be brine, a water-containing hydrocarbon, a water-containing gas, or a combination thereof. The corrosion inhibitor composition can be introduced into the environment in an amount in the range from about 1 to about 1000 parts per million by volume. The corrosion inhibitor composition can also be introduced into the environment in an amount of about 1000 parts per million by volume or less.

Various illustrative embodiments of a composition for inhibiting corrosion of a metal in an environment in which the metal is corrodible are also provided herein. In certain aspects, the composition can include 5-amino-2-mercaptobenzimidazole. The metal can be carbon steel.

### BRIEF DESCRIPTION OF THE DRAWINGS

FIG. 1 is a performance comparison graph (corrosion rate over time) for 5-amino-2-mercaptobenzimidazole and 2-MEBI in an illustrative embodiment.

While certain preferred illustrative embodiments will be described herein, it will be understood that this description is not intended to limit the subject matter to those embodiments. On the contrary, it is intended to cover all alternatives, modifications, and equivalents, as may be included within the spirit and scope of the subject matter as defined by the appended claims.

### DETAILED DESCRIPTION

Various illustrative embodiments of a composition for inhibiting corrosion of metals are disclosed herein. Corrosion of both ferrous and non-ferrous metals, induced by a variety of corrosive aqueous-based environments, may be inhibited or controlled through use of the corrosion inhibiting composition disclosed herein. The corrosion inhibiting composition can inhibit corrosion to a degree that is comparable to or significantly greater than the inhibition provided by equal amounts of certain prior corrosion inhibitor compounds, particularly at low inhibitor concentrations. The corrosion inhibitor composition would be particularly suitable for use on metals in a hydrocarbon production system.

In certain illustrative embodiments, the corrosion inhibitor composition comprises 5-amino-2-mercaptobenzimidazole. 5-amino-2-mercaptobenzimidazole has the chemical formula C₇H₇N₃S and the following structure:

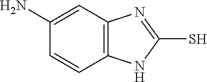 [SMILES: Nc1ccc2[nH]c(S)nc2c1]

5-amino-2-mercaptobenzimidazole is commercially available from, among other sources, Sigma-Aldrich of St. Louis, Mo. 5-amino-2-mercaptobenzimidazole may also be prepared by any means and methods known to those skilled in the art.

In certain illustrative embodiments, an amino group has been added to 2-MEBI to form 5-amino-2-mercaptobenzimidazole. Without wishing to be bound by theory, it is believed that addition of the amino group greatly increases the inhibition efficiency of the molecule, such that it outperforms many other sulfur-containing corrosion inhibitors in standard kettle corrosion testing.

In certain illustrative embodiments, the metal that is treated with the corrosion inhibitor composition can comprise carbon steel. However, many other ferrous and non-ferrous metals, including alloys, can be effectively treated with the corrosion inhibitor composition, including, for example, aluminum, iron, zinc, nickel, and copper, and combinations of these. In general, the corrosion inhibiting composition disclosed herein may be used for the purpose of inhibiting corrosion for any ferrous or non-ferrous metals, in both elemental and alloyed form. A particular type of metal that would be suitable for treatment includes steel tubulars, pipelines, and equipment used in the production of oil and gas.

In certain illustrative embodiments, a method of inhibiting corrosion of a metal is also provided wherein the metal is contacted with a corrosion inhibitor composition comprising an effective amount of 5-amino-2-mercaptobenzimidazole. The corrosion inhibitor composition may also contain other components or materials, such as, for example, primary amines, secondary amines, tertiary amines, quaternary amines, imidazoline derivatives, phosphate derivatives, thiol derivatives, pyridine derivatives, organic acids, fatty acids, alkyl alcohols, surfactants, oxygen scavengers and scale inhibitors. Solvents or diluents may also be employed together with the corrosion inhibitor composition of this invention, which solvents may include, but are not necessarily limited to, water, alcohols, aromatic solvents, such as naphthas and xylene, and the like. In certain illustrative embodiments, the proportion of 5-amino-2-mercaptobenzimidazole in the composition may range from about 0.1 to about 80 weight %. Preferably, the proportion of 5-amino-2-mercaptobenzimidazole in the composition ranges from about 1 to about 60 weight %.

In certain illustrative embodiments, the corrosion inhibitor composition is contacted with the metal in an environment where the metal would otherwise be corrodible therein. For example, the environment can comprise a corrosive aqueous-based environment such as brine, water-containing hydrocarbons, water-containing gases, or combinations thereof.

In certain illustrative embodiments, a method of inhibiting corrosion of a metal is also provided wherein the metal is contacted with an effective amount of a corrosion inhibitor composition comprising 5-amino-2-mercaptobenzimidazole.

In general, the corrosion inhibitor composition can be contacted with the metal in an amount sufficient to provide the desired corrosion-reducing properties. The corrosion inhibitor composition is particularly effective at low dosage, that is, low inhibitor concentrations. These concentrations can be based on water only, or on total fluids. For example, the corrosion inhibitor composition can be applied in an amount in the range of from about 1 to about 1000 ppmv (parts per million by volume), in certain illustrative embodiments. These concentration values are at, or above, the minimum effective concentration (MEC) for the corrosion inhibitor composition. The corrosion inhibitor composition can also be applied in an amount of about 1000 ppmv or less (parts per million by volume), in certain illustrative embodiments. If a system was dosed with the corrosion inhibitor composition based on total fluids, then the effective dosage in the water could be significantly higher than 1 ppmv depending on the partitioning coefficient (considering a 1% water cut where all of the corrosion inhibitor composition went into the water). In the case where the corrosion inhibitor composition partitioned highly to the water, much lower dosages based on total fluids could also be effective, in certain illustrative embodiments.

In most oil and gas production systems, the amount of chemical that can be injected to prevent corrosion is limited by the system design. For instance, on an offshore platform the umbilical that is used to deliver chemical subsea might be limited to 100 gal/hr of maximum injection rate. If the platform was producing 100,000 bbl/day, then the system would need to be designed to inject 175 gal/hr to dose 1000 ppmv. Therefore, this system would be underdosed by 75 gal/hr (˜43%). Also, for an offshore platform there may be storage limitations. The storage limitations and delivery schedules offshore might limit the volume of corrosion inhibitor that can be applied on that platform.

To facilitate a better understanding of the presently disclosed subject matter, the following examples of certain aspects of certain embodiments are given, as compared to prior art systems. In no way should the following examples be read to limit, or define, the scope of the presently disclosed subject matter.

Experimental Testing

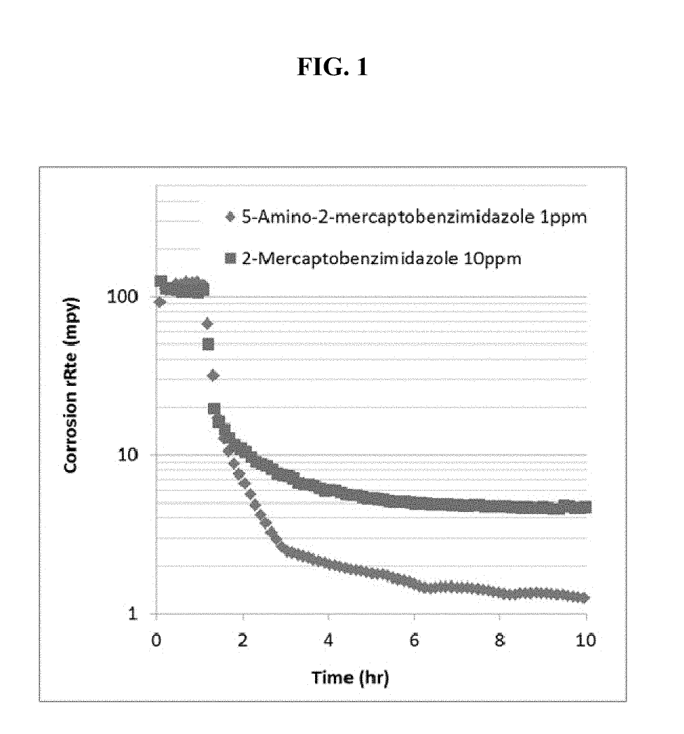

A performance comparison was conducted for 5-amino-2-mercaptobenzimidazole and 2-MEBI, which is a corrosion inhibitor product commercially available from Baker Hughes Incorporated. The tests were conducted at 1 ppm dosage for both inhibitors. The kettle corrosion testing was performed using a standard 1 L resin kettle at 180° F. with a 90% synthetic brine solution which has 94 g/L NaCl, 4.1 g/L CaCl₂ and 1.9 g/L MgCl₂, and 10% ISOPAR™ M. The kettle was sparged with 100% CO₂ at 100 mL/min. In this test, a lower corrosion rate beyond one hour after chemical injection is indicative of better corrosion inhibition performance. The results of the performance comparison are shown in FIG. 1 herein. 5-amino-2-mercaptobenzimidazole has markedly better performance than 2-MEBI beyond one hour after injection. The inhibitor would work well at higher concentrations as well. Once the minimum effective concentration (MEC) is reached with an intermediate, the performance does not improve significantly with increasing concentration. The testing indicated that the corrosion inhibitor product was over the MEC for this intermediate at 1 ppm dosage, so no testing at higher concentrations was needed.

While the disclosed subject matter has been described in detail in connection with a number of embodiments, it is not limited to such disclosed embodiments. Rather, the disclosed subject matter can be modified to incorporate any number of variations, alterations, substitutions or equivalent arrangements not heretofore described, but which are commensurate with the scope of the disclosed subject matter. Additionally, while various embodiments of the disclosed subject matter have been described, it is to be understood that aspects of the disclosed subject matter may include only some of the described embodiments. Accordingly, the disclosed subject matter is not to be seen as limited by the foregoing description, but is only limited by the scope of the appended claims.

What is claimed is:

1. A method of inhibiting corrosion of a metal, the method comprising contacting the metal with a corrosion inhibitor composition comprising an effective amount of 5-amino-2-mercaptobenzimidazole, wherein the metal comprises iron.

2. (canceled)

3. The method of claim 1, wherein the corrosion inhibitor composition is contacted with the metal in an environment where the metal would otherwise be corrodible therein.

4. The method of claim 3, wherein the environment is brine, a water-containing hydrocarbon, a water-containing gas, or a combination thereof.

5. The method of claim 4, wherein the corrosion inhibitor composition is introduced into the environment in an amount in the range from about 1 to about 1000 parts per million by volume.

6. The method of claim 4, wherein the corrosion inhibitor composition is introduced into the environment in an amount of about 1000 parts per million by volume or less.

7.-12. (canceled)

13. A composition for inhibiting corrosion of a metal in an environment in which the metal would otherwise be corrodible, the composition comprising 5-amino-2-mercaptobenzimidazole, wherein the metal comprises iron.

14. (canceled)

15. The method of claim 1, wherein the metal comprises an iron alloy.

16. The method of claim 3, wherein the environment comprises one or more of CO₂ and H₂S.

17. The composition of claim 13, wherein the metal comprises an iron alloy.

18. The composition of claim 13, wherein the environment comprises one or more of CO₂ and H₂S.
